(19) United States
(12) Patent Application Publication
(10) Pub. No.: US 2002/0072627 A1
(43) Pub. Date: Jun. 13, 2002

(54) Process for the manufacture of alkali metal salt of sorbic acid or free Sorbic Acid by oxidation

(75) Inventors: S. B. Chandalia, Mumbai (IN); Kavita Chandnani, Mumbai (IN); Sangeeta Srivastawa, Mumbai (IN)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Somaiya Organo Chemicals Limited

(21) Appl. No.: 09/982,099

(22) Filed: Oct. 19, 2001

(30) Foreign Application Priority Data
Oct. 25, 2000 (IN) 957/MUM/2000

(51) Int. Cl. C07C 51/235

(52) U.S. Cl. 562/531

(57) ABSTRACT

A process for the manufacture of alkali metal salt of Sorbic acid or free Sorbic acid by oxidation of 2:4 hexadienal with an aqueous solution of Hydrogen Peroxide and adding oxides of transition metal such as Mn, Ni, Cu, Ag, Pt, or Pd. In a reactor maintaining the temperature in the range of 5 deg.-80 deg.C., preferable between 20 deg. to 40 deg. thus converting the metal salt to free Sorbic acid by acidification.

Molar proportions of 2:4 hexadienal and an aqueous alkali metal hydroxide are 1:2:1 and these are fed to reactor containing catalyst and continuously blowing the mixture.

Catalysts used in the process are oxides of transition metals or salts of Li, Na, or Ca.

The mixture in the reactor kept alkaline using calcium hydroxide or ammonium hydroxides. Potassium hydroxide or ammonium hydroxides.

The present invention relates to a process for the manufacture of alkali metal salt of Sorbic acid or free Sorbic Acid by oxidation.

### DESCRIPTION

Sorbic Acid and its salts have been preferably used as preservatives for foods because of their excellent antifungal activity, various processes are known for the preparation of Sorbic Acid. e.g. condensation of the malonic acid with Crotonaldelyde, the reaction of crotonaldehyde with zinc derivative of acetic bromine ester as per REFORMATZKY. Synthesis, oxidation of hexadienal with silver salts, the oxidation of 3, 5, heptadienone with Sodium hypochlorite and others.

Sorbic Acid has been industrially manufactured by reacting crotonaldehyde with ketene to give a polyster through a lactone produced as an intermediate and hydrolysing the polyester with an alkali or an acid or by using an ion exchange resin. However, the above mentioned process requires a high input of energy during the cracking of acetic acid to ketene and it is not advantageous economically. Further recovery and purification of Sorbic Acid entails several steps and involves complicated process controls.

Unsaturated organic acids are prepared by the oxidation of corresponding aldehydes with air. As these are unsaturated the oxidation frequently becomes very difficult because of the formation of by-products which reduce the yield of end product and adversely affect the purity of the final product.

U.S. Pat. Specification No. 3,313,843 describes the conversion of ethylenic aldehydes into corresponding acid using Nickel Peroxide in an alcoholic medium. The use of Stoichiometric amount of Nickel Peroxide would not be cost effective. U.S. Pat. Specification No. 2,887,496 and Indian patent Specification No. 139231 described the oxidation of aldehyde with air, in the presence of a metal catalyst such as silver and an aqueous solution of an alkaline reagent. In this case the desired acid is obtained in the form of an alkali metal salt, and one has to thereafter acidify the salt to liberate the acid.

The yield of crude acid is good but the amount of catalyst employed is relatively large and the acid obtained is a crude product which must be purified by washing recrystallisation and stripping—French Patent Specification No. 1,427,301 describes the oxidation of unsaturated aldehydes with air in the presence of Palladium salt and Copper halide or Iron halide.

It is a well-known fact that recurring expenditure is high as molecular oxygen is used as oxidant. Further under the conditions used the oxidation of hexadienal is accompanied by many side reactions resulting Polymeric by Products. This process is use of molecular oxygen, therefore, does not produce the desired product.

As an Indian Patent Specification No. 139231, it is observed that by oxidation of aldehyde a lot of by-products are formed and yield is poor (54% to 74%) During oxidation other isomer of sorbic acid i.e. cis-2 trans-4-lexadienoic acid is formed which is difficult to convert into trans-2-, trans-4, hexadicnoic acid. For these reasons the above processes are expensive,uneconomical and poor in yield.

The object of the present invention is to provide a process for the manufacture of alkali metal salt of Sorbic acid or free Sorbic acid by oxidation using reagents such as hydrogen peroxide or alkyl peroxide in the presence of metal oxides or hydroxides as a catalyst.

This is commercial viable process for producing Sorbic Acid, from Sorbaldehyde (2:4-hexadienal), in greater yields and high degree of purity.

### SUMMARY OF THE INVENTION

A process for the manufacture of alkali metal salt of Sorbic acid or free Sorbic acid by oxidation of 2:4 hexadienal with an aqueous solution of Hydrogen Peroxide and adding oxides of transition metal such as Mn, Ni, Cu, Ag, Pt, or Pd. In a reactor maintaining the temperature in the range of 5 deg.—80 deg.C., preferable between 20 deg. to 40 deg. thus converting the metal salt to free Sorbic acid by acidification.

Molar proportions of 2:4 hexadienal and an aqueous alkali hydroxide are 1:2:1 and these are fed to reactor containing catalyst and continuously blowing the mixture.

Catalysts used in the process are oxides of transition metals or salts of Li, Ma, or Ca.

The mixture in the reactor kept alkaline using calcium hydroxide or ammonium hydroxides, Potassium hydroxide or ammonium hydroxides.

Aqueous Solution of methanol ethanol, propanol, butalol, petanol or decanol were preferred for enhancing the rate of reaction.

The contents of the reaction are filtered and acidified with hydrochloric acid yielding 92% to 98% pure sorbic acid.

Metal oxides or hydroxides such as silver oxide is used as catalyst. The invented process relevant to aldehydes where the molecule does not withstand the usual treatment of oxidation with air because of carbon-carbon unsaturation in the molecule. It is also especially relevant to ethylenic and polyenic, aliphatic, aromatic aldehydes such as acrolein, methaacrolein, crotonaldehyde, sorbaldehyde, cinnamaldehye and their homologue.

The reaction is carried out in an aqueous solution of an inorganic alkaline reagent. This reagent must be an alkali metal hydroxide such as sodium hydroxide or potassium hydroxide. The amount of alkaline reagent must be sufficient to convert the acid into salt at the rate at which it is formed; a molar ratio of alkali metal hydroxide/aldehyde of 1:1:2.

Extensive investigations have been carried out for discovering that when aqueous hydrogen peroxide or alkylhydroperoxide or acyl peroxide is used as an oxidant, the conversion and selectivity can be improved and moreover the resulting sorbic acid has a low content of impurities causing discolouration and thus decreases the load on subsequent purification steps and thus the consequent yield and quality and purity of sorbic acid increases considerably for manufacturing sorbic acid and its derivatives over all other conventional processes.

### DESCRIPTION OF THE INVENTION

The present invention relates to the oxidation of unsaturated aldehydes using oxidising agents that may be sodium hypohalites such as hypochlorites, or directly hypohalous acids or their salts e.g. lithium, sodium or calcium, peroxides such as benzoyl peroxide, hydrogen peroxide, acetyl peroxide, alkyl hydro peroxides such as amyl hydroperoxide. It was observed during the discovery that peracids such as peracetic acid, perbenzoic acid, mono perthalic acid or m-chloro perbenzoic acid also gave very good yields to the corresponding acid but the alkali requirement was high.

The catalysts used for reaction are oxides of transition metals as Mn, Ni, Cu, Ag, Pt or Pd. It was found that group IB and VIII elements gave good results. The alkali used for the reaction may be calcium hydroxide, sodium hydroxide, sodium carbonate, potassium hydroxide, ammonium hydroxide. It was also found that organic bases were also equally effective with the advantage of lesser polymerisation rates than the inorganic bases. Amines such as triethanol emines, mono. di-alkyl anines. EDFA etc. increased the selectively to the corresponding acid.

To increase the rate of the reaction the use of aquieous alcoholic solution was favourable. Alkanols such as methonol ethanol, propanol, butanol, pentanol or decanol were preferred.

Temperature was kept in the rate of 5-80° and preferably between 20 to 40°C. The aldehyde and the catalyst was taken in a suitable aqueous solvent in the reactor while the oxidisingy agent and alkali were added, separately, drop-wise in a period of 4-24 h.

The following examples are illustrative of the present invention.

### Example 1

500 ml of water and 100 gm of 2,4-hexadienal from the plant containing 5 gm of CoO were taken in a 1000 cm³ reactor. The reaction contents were stirred at 1200 rpm at 50°C. 200 ml of 12% sodium hypochlorite and 80 gm of sodium carbonate were added in 10 Hrs.

Instead of taking CoO directly better yields were obtained by preparing the catalyst in situ. The catalyst is prepared by reacting cobalt nitrate with equimolar amounts of sodium hypochlorite. The solution were continuously stirred. The solution was further filtered and soon after filtrate was acidified with 20% hydrochloric acid Yield of 72.9 gm of sorbic acid with 94% was obtained.

### Example-2

500 ml water and 300 ml of ethanol were mixed and 100 g of sorbaldehyde with 2% w/v catalyst loading of CuO was taken in reactor. 50 g of benzoyl peroxide and 59 g potassium hydroxide were added at 42 C in 8 h. Work-up was done as in example 1.84 g of sorbic acid of 95.2 purity was obtained.

### Example-3

300 ml water and 200 ml of propanol were mixed and 100 g of sorbaldehyde with 5% NiO2 was taken in reactor. 50 g of benzoyl peroxide and 77 g potassium hydroxide were added at 42° C. in 8 h. Work-up was done as in example 1.81 g of NiO2 prepared in situ by nickel sulfate and sodium hydroxide gave about 5% higher yields.

### Example-4

475 ml water and 25 ml o methanol were mixed and 100 g of sorbaldehyde with a catalyst loading of 100% MnO2 were taken in reactor. 50 g of benzoyl peroxide and 50 g potassium hydroxide were added at 57° C. in 5.5 h. Work-up was done as in example 1.70.6 g of sorbic acid of 97.6% purity was obtained.

### Example-5

25 ml water-and 475 ml of propanol were mixed and 100 g of sorbaldehyde with a catalyst loading of 3% PtO2 were taken in reactor. 200 ml of hydrogen peroxide and 55 ml of triethanol amine were added separately at 77° C. in 12 h. Work-up was done as in example 1.67.6 g of sorbi of 94.6% purity was obtained.

### Example-6

500 ml water and 100 g of sorbaldehyde with 5% NiO2 were taken in a reactor. 90 g of pyridine N-oxide was added. In this case there was no need to add alkali as pyridine liberated after reaction acts as the base.

Acidification was done with 30% HCL.

74 g of sorbic acid of 92.3% purity was obtained.

### Example-7

500 ml water and 100 g of sorbaldehyde with 5% Ag2O were taken in a reactor. 90 g of Hydrogen peroxide was added. In this case there was need to add alkali as pyridine liberated after reaction acts as the base.

Acidification was done with 30% of HCL.

85 g of sorbic acid of 99.3% purity was obtained.

We claim:

1.) A process for the manufacture of alkali metal salt of sorbic acid or free sorbic acid by oxidation of 2:4 hexadienal with in aqueous solution of Hydrogen peroxide as an oxidising agent and silver oxide as catalyst in a reactor.

2.) A process as claimed in claim-1 in which molar proportions of 2:4 hexadienal and aqueous alkali metal hydroxide are 1.2:1.

3.) A process as claimed in claims-1 and 2 wherein molar proportions of 2:4 hexadienal and aqueous alkali metal hydroxide are separately fed to a reactor containing catalyst and stirring the mixture continuously.

4.) A process as claimed in claim-3 wherein catalysts used are oxides of transition metal—Mn, Ni, Cu, Ag, Pt or Pd.

5.) A process as claimed in claims-1, 2 and 3 in which reactants are thoroughly and continuously mixed through the reaction in a period of 4-24 Hrs.

6.) A process as claimed in claims 1 to 5 in which the temperatures of the reaction should be maintained in the range of 5° to 80° C., preferably between 20° to 40° C.

7.) A process as claimed in previous claims wherein the reaction mixture is kept alkaline.

8.) A process as claimed in claim-7 wherein the alkali used are Calcium hydroxide, Sodium hydroxide, Potassium hydroxide or Ammonium hydroxide.

9.) A process as claimed in previous claims, aqueous solution of methanol, ethanol, propanol, butanol, pentanol or decanol were preferred for enhancing the rate of reaction.

10.) A process as claimed in any previous claims wherein the contents of the reaction are filtered to free the product and acidified with hydrochloric acid and yieding 92% to 99.3% pure sorbic acid.

11.) A process for the manufacture of alkali metal salt of sorbic acid or free sorbic acid substantially as herein described and with reference to Examples 1 to 6.
